(19) United States
(12) Patent Application Publication
(10) Pub. No.: US 2026/0185935 A1
(43) Pub. Date: Jul. 2, 2026

(54) DETERMINING A COFFEE ROAST SCORE

(71) Applicant: Kraft Foods Group Brands LLC, Chicago, IL (US)

(72) Inventor: Kevin Anthony Wong, Barrington, IL (US)

(21) Appl. No.: 19/393,835

(22) Filed: Nov. 19, 2025

Related U.S. Application Data
(60) Provisional application No. 63/739,781, filed on Dec. 30, 2024.

(51) Int. Cl. G01N 21/359; G01N 21/31; G01N 21/3563; G01N 33/02

(52) U.S. Cl. CPC G01N 21/359; G01N 21/314; G01N 21/3563; G01N 33/025; G01N 2021/3148

(57) ABSTRACT

A coffee bean sample is exposed to near-infrared light followed by determining a reflected near-infrared light spectrum from the coffee bean sample. A roast score for the coffee bean sample is then determined using some, but not all, frequencies between 8000 wavenumbers and 4000 wavenumbers in the reflected near-infrared light spectrum. By one approach, determining the roast score for the coffee bean sample further comprises also using some, but not all, frequencies between 12,000 wavenumbers and 4,000 wavenumbers aside from the frequencies that are used between 8000 wavenumbers and 4000 wavenumbers.

### CROSS-REFERENCE TO RELATED APPLICATIONS

This application claims the benefit of U.S. Provisional Application No. 63/739,781, filed Dec. 30, 2024, which is incorporated herein by reference in its entirety.

### TECHNICAL FIELD

These teachings relate generally to roasting coffee beans and more particularly to scoring coffee roast.

### BACKGROUND

Coffee beans are roasted to develop flavor, aroma, and color. Green coffee beans have very little of the complex flavors and aromas that make coffee enjoyable. Generally speaking, light roasts tend to retain more of the beans'original acidity, while darker roasts usually have less acidity and a more pronounced bitterness.

The Agtron roast score is a proprietary scale ranging from 0 to 100 that serves as an industry standard for assessing the degree of roast in coffee beans. The Agtron™ coffee roast analyzer uses near-infrared spectroscopy to determine the degree of roast by measuring the reflectance of ground coffee or whole coffee beans. The resulting data is converted into a so-called Agtron number on the aforementioned scale.

Unfortunately, current approaches in these regards can nevertheless be unduly subjective and/or inconsistent. This is due, at least in part, to the design of existing testing instruments that can make repeatability uncertain and with the judgement of the human analyst often playing an important role. Some research suggests that the foregoing approach has a root mean square error of 3.67.

### BRIEF DESCRIPTION OF DRAWINGS

The above needs are at least partially addressed through provision of the approach to determining a coffee roast score described in the following detailed description, particularly when studied in conjunction with the drawings, wherein:

FIG. 1 comprises a block diagram as configured in accordance with various embodiments of these teachings;

FIG. 2 comprises a graph as configured in accordance with various embodiments of these teachings; and

FIG. 3 comprises a graph as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments, a coffee bean sample is exposed to near-infrared light followed by determining a reflected near-infrared light spectrum from the coffee bean sample. A roast score for the coffee bean sample is then determined using some, but not all, frequencies between 8000 wavenumbers and 4000 wavenumbers in the reflected near-infrared light spectrum.

By one approach, determining the roast score for the coffee bean sample further comprises also using some, but not all, frequencies between 12,000 wavenumbers and 4,000 wavenumbers aside from the frequencies that are used between 8000 wavenumbers and 4000 wavenumbers.

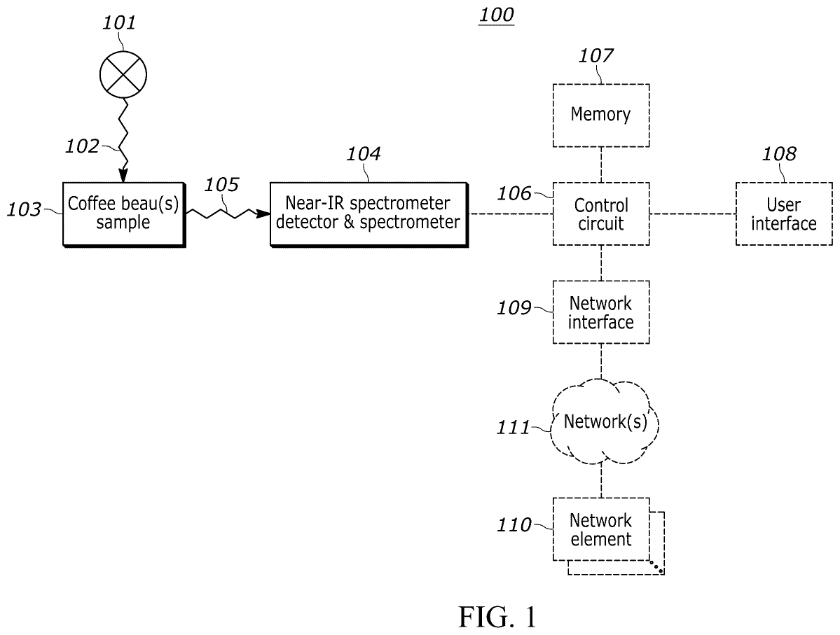

Various benefits may become clearer upon making a thorough review and study of the following detailed description. Referring now to the drawings, and in particular to FIG. 1, an illustrative apparatus 100 that is compatible with many of these teachings will first be presented.

These teachings employ near-infrared spectroscopy, which is a non-invasive optical technique used to measure the absorption of near-infrared light by chromophores (molecules that can absorb light) in a sample. It operates in the near-infrared region of the electromagnetic spectrum, which ranges from about 780 nm to about 2500 nm (0.78 to 2.5 micrometers). Near-infrared spectroscopy leverages the fact that different chemical compounds absorb light at specific wavelengths, based on their molecular structure and bonding.

The basic setup for near-infrared spectroscopy involves a light source 101 that emits near-infrared light 102 directed towards a sample (in this case, a roasted coffee bean sample 103), a detector 104 that measures the intensity of light 105 that has passed through or been reflected from the sample, and a spectrometer (also 104) or similar device that analyzes the wavelengths of the light 105 to determine the absorption characteristics of the sample. (For convenience, as used herein, the expression “coffee bean” will be understood to refer to either unground coffee beans or ground coffee beans.)

In this particular example, the enabling apparatus 100 includes a control circuit 106. This control circuit 106 may be separate from the aforementioned components (as illustrated) or may be, in whole or in part, integral to, for example, the aforementioned spectrometer 104.

Being a “circuit,” the control circuit 106 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 106 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 106 is configured (for example, by using corresponding programming as will be well understood by those skilled in the art) to carry out one or more of the steps, actions, and/or functions described herein.

It will be appreciated that the control circuit 106 may comprise a single integrated platform or may comprise a plurality of such circuits that work in cooperation with one another.

The control circuit 106 can operably couple to a memory 107. This memory 107 may be integral to the control circuit 106 or can be physically discrete (in whole or in part) from the control circuit 106 as desired. This memory 107 can also be local with respect to the control circuit 106 (where, for example, both share a common circuit board, chassis, power supply, and/or housing) or can be partially or wholly remote with respect to the control circuit 106 (where, for example, the memory 107 is physically located in another facility, metropolitan area, or even country as compared to the control circuit 106). As with the control circuit 106, the memory 107 may comprise a singular structure or may comprise a plurality of memory platforms that collectively comprise the “memory” of this apparatus 100.

In addition to information such as historical information for previous sample readings, this memory 107 can serve, for example, to non-transitorily store the computer instructions that, when executed by the control circuit 106, cause the control circuit 106 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 106 also operably couples to a user interface 108. This user interface 108 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 106 can also operably couple to a network interface 109. So configured the control circuit 106 can communicate with other network elements 110 (such as, for example, other user interfaces where information regarding the analysis of a particular sample of coffee beans can be viewed by a user) via one or more intervening communication networks 111 (such as, but not limited to, the Internet).

Centimeters to the negative first power (cm⁻¹) is a unit often used to express wavenumbers, which are the spatial frequency of a wave such as a wave of near-infrared light. The wavenumber relates to the wavelength (λ) as a reciprocal thereof. Wavenumbers are commonly used in spectroscopy to represent the energy levels of molecular vibrations and other phenomena.

The applicant notes that near-infrared spectrometry has been previously used to measure a degree of coffee bean roast, and in fact underlies the current approach to develop an Agtron roast score. That prior art approach uses the region of 8,000 to 4,000cm⁻¹. As noted above, however, prior art research indicates that this prior art approach has a root mean square error value of 3.67.

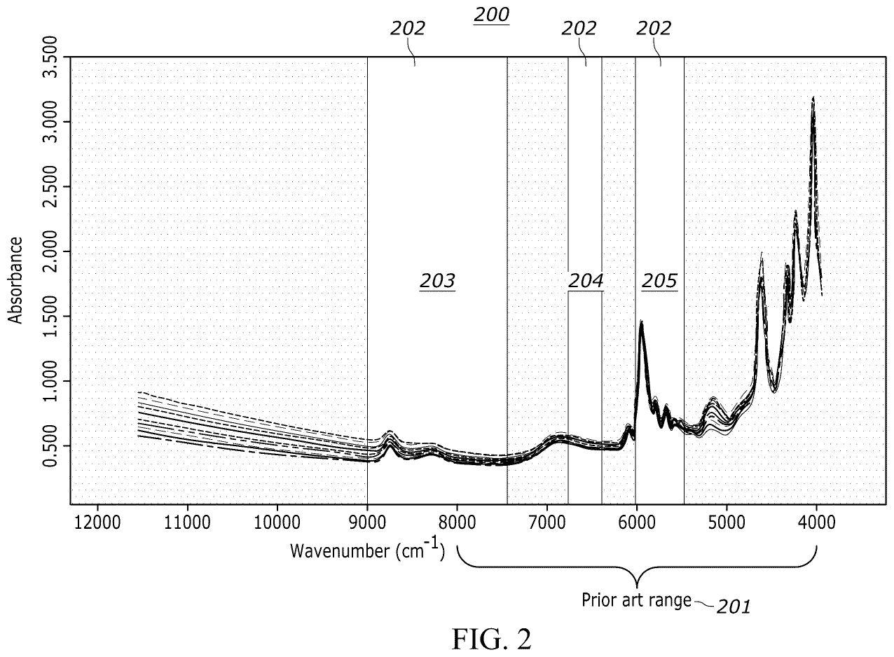

Like the prior art approach, the applicant uses near-infrared spectrometry. The applicant has determined, however, that better accuracy can be achieved by making a more selective use of the near-infrared light spectrum. FIG. 2 presents a graph 200 of that spectrum. The X-axis represents wavenumbers while the Y-axis represents absorbance. The prior art utilized range is denoted by reference numeral 201.

The applicant's approach utilizes a part of that prior art range 201, but only a part. In addition, the applicant also utilizes a part, but only a part, of the overall near-infrared spectrum that is outside the prior art range 201.

The portions utilized by the applicant are denoted by reference numeral 202.

These included portions comprise a first region 203 that corresponds to 9000-7448 cm⁻¹. This region includes carbon-hydrogen stretch second overtones and cis-double bonds (where cis-double bonds refers to the geometric configuration of a double bond in an alkene). A second region 204 corresponds to 6776-6400 cm⁻¹ and includes typical first overtone bonds of bonded OH—H bonding (and hence, hydrogen bonding of water). And a third region 205 that corresponds to 6032-5496 cm⁻¹ and includes carbon-hydrogen stretch first overtones and methyl groups from cellulose or lignin.

The applicant determined to select the foregoing frequencies to assess a roast score, at least in part, because the selected regions demonstrated a higher amount of variation that correlates with Agtron values. The applicant believes that the prior art frequency range may not consistently correlate as well with the Agtron roast score. When viewed from a chemical standpoint, as coffee beans are roasted there is an accompanying change in the structure of the fibers in the coffee beans as well as a change in the distribution of moisture. The applicant believes it possible that the selected ranges are better suited to leverage that phenomenon.

By one approach, these teachings will accommodate filtering out sample-based light in the unused frequency ranges while retaining the selected frequency ranges when calculating the roast score using otherwise traditional calculations.

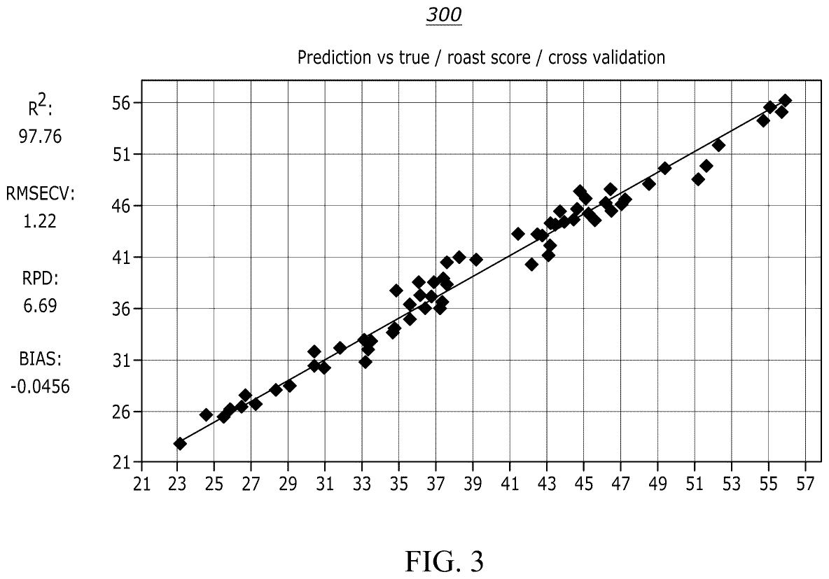

In any event, FIG. 3 presents a graph 300 that depicts a number of (predicted) roast score values that were determined when using the applicant's selected frequency ranges as compared to corresponding true scores. The assessed scores appear on the X axis while the true scores appear on the Y axis. Over a considerable useful range of roast scores it can be seen that the root mean squared error value was only 1.23, a considerable improvement in consistent accuracy as compared to the prior art.

During ordinary use, coffee beans 103 may be sampled following roasting (or, in a suitable application setting, intermittently or continuously during the roasting process) by, for example, the aforementioned control circuit 106. The generated roast score may then be stored in memory (for example, for archival purposes), used in a feedback loop to determine when the batch of coffee beans 103 have been roasted to a desired degree, and/or reported to one or more human users via, for example, the aforementioned user interface 108.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method comprising:
exposing a coffee bean sample to near-infrared light;
determining a reflected near-infrared light spectrum from the coffee bean sample;
determining a roast score for the coffee bean sample using some, but not all, frequencies between 8000 wavenumbers and 4000 wavenumbers in the reflected near-infrared light spectrum.

2. The method of claim 1 wherein determining the roast score for the coffee bean sample further comprises also using some, but not all, frequencies between 12,000 wavenumbers and 4,000 wavenumbers aside from the frequencies that are used between 8000 wavenumbers and 4000 wavenumbers.

3. The method of claim 1 wherein determining a roast score for the coffee bean sample using some, but not all, frequencies between 8000 wavenumbers and 4000 wavenumbers in the reflected near-infrared light spectrum comprises using at least two separate and discrete frequency bands.

4. The method of claim 3 wherein using at least two separate and discrete frequency bands comprises using at least three separate and discrete frequency bands.

5. The method of claim 4 wherein using at least three separate and discrete frequency bands comprises using exactly three separate and discrete frequency bands.

6. The method of claim 4 wherein two of the separate and discrete frequency bands are located between 8000 wavenumbers and 4000 wave numbers while a third one of the separate and discrete frequency bands is at least partially located outside that 8000 wavenumbers to 4000 wavenumbers range.

7. An apparatus comprising:
a control circuit configured to:
receive reflected near-infrared light spectrum from a coffee bean sample;
determine a reflected near-infrared light spectrum from the coffee bean sample;
determine a roast score for the coffee bean sample using some, but not all, frequencies between 8000 wavenumbers and 4000 wavenumbers in the reflected near-infrared light spectrum.

8. The apparatus of claim 7 wherein the control circuit is configured to determine the roast score for the coffee bean sample by also using some, but not all, frequencies between 12,000 wavenumbers and 4,000 wavenumbers aside from the frequencies that are used between 8000 wavenumbers and 4000 wavenumbers.

9. The apparatus of claim 7 wherein the control circuit is configured to determine a roast score for the coffee bean sample using some, but not all, frequencies between 8000 wavenumbers and 4000 wavenumbers in the reflected near-infrared light spectrum by using at least two separate and discrete frequency bands.

10. The apparatus of claim 9 wherein the control circuit is configured to use at least two separate and discrete frequency bands by using at least three separate and discrete frequency bands.

11. The apparatus of claim 10 wherein the control circuit is configured to use at least three separate and discrete frequency bands by using exactly three separate and discrete frequency bands.

12. The apparatus of claim 10 wherein two of the separate and discrete frequency bands are located between 8000 wavenumbers and 4000 wave numbers while a third one of the separate and discrete frequency bands is at least partially located outside that 8000 wavenumbers to 4000 wavenumbers range.
